(19) United States
(12) Patent Application Publication
(10) Pub. No.: US 2013/0338534 A1
(43) Pub. Date: Dec. 19, 2013

(54) TISSUE EXTRACTION DEVICE

(71) Applicants: John P. Cole, Alpharetta, GA (US); Tesfaye G. Habtemariam, Alpharetta, GA (US)

(72) Inventors: John P. Cole, Alpharetta, GA (US); Tesfaye G. Habtemariam, Alpharetta, GA (US)

(21) Appl. No.: 13/921,013

(22) Filed: Jun. 18, 2013

Related U.S. Application Data
(60) Provisional application No. 61/661,270, filed on Jun. 18, 2012.

(51) Int. Cl. A61B 10/06

(52) U.S. Cl. CPC A61B 10/06
USPC 600/571; 600/570

(57) ABSTRACT

A tissue extraction device. The tissue extraction device includes a first handle, the first handle terminating in a first tip. The tissue extraction device also includes a second handle. The second handle is coupled to the first handle and terminates in a second tip. The tissue extraction device further includes a tissue collection mechanism, where the tissue collection mechanism is configured to collect tissue extracted from a patient by the first tip and the second tip.

### CROSS-REFERENCE TO RELATED APPLICATIONS

This application claims the benefit of and priority to U.S. Provisional Patent Application Ser. No. 61/661,270 filed on Jun. 18, 2013, which application is incorporated herein by reference in its entirety.

### BACKGROUND OF THE INVENTION

During various surgical procedures, extraction of tissue specimens during the procedure may be necessary. For example, the tissue may need to be tested or otherwise examined. One such procedure is a follicular dissection and transplantation. During a follicular dissection, the careful extraction of hair follicle grafts is implemented in order to ensure successful transplantation of hair follicles. Follicular dissection and extraction is accomplished by gently isolating hair follicles and skillfully extracting them from a donor site with the help of a tissue extracting device.

Another surgical procedure requiring processing of harvested tissue is strip hair transplantation which is strip harvesting of skin tissue having hair follicles from one area of the body for transplantation to another area of the body. Here a surgeon excises tissue in the donor area using a surgical scalpel to release tissue and may extract the tissue with a specialized forceps or other tissue extraction device. The strip may then be processed and transplanted to the desired area.

However, many tissue extraction devices tend to be ill suited for these uses. In particular, the tissue extraction devices tend to remove large pieces of tissue, causing pain and/or making the tissue unsuitable for transplantation. In addition, the tissue extraction devices lack a collection mechanism for the extracted tissue. Thus, the tissue may be lost.

Accordingly, there is a need in the art for a tissue extraction device which can remove only the desired tissue. Further, there is a need in the art for a tissue extraction device which can collect the extracted tissue.

### BRIEF SUMMARY OF SOME EXAMPLE EMBODIMENTS

This Summary is provided to introduce a selection of concepts in a simplified form that are further described below in the Detailed Description. This Summary is not intended to identify key features or essential characteristics of the claimed subject matter, nor is it intended to be used as an aid in determining the scope of the claimed subject matter.

One example embodiment includes a tissue extraction device. The tissue extraction device includes a first handle, the first handle terminating in a first tip. The tissue extraction device also includes a second handle. The second handle is coupled to the first handle and terminates in a second tip. The tissue extraction device further includes a tissue collection mechanism, where the tissue collection mechanism is configured to collect tissue extracted from a patient by the first tip and the second tip.

Another example embodiment includes a tissue extraction device. The tissue extraction device includes a first handle, the first handle including a first guide and terminating in a first tip. The first tip includes denticulations. The tissue extraction device also includes a second handle. The second handle is coupled to the first handle and terminates in a second tip, where the second tip includes denticulations. The second handle includes a second guide configured to mate with the first guide. The tissue extraction device further includes a tissue collection mechanism, where the tissue collection mechanism is configured to collect tissue extracted from a patient by the first tip and the second tip.

Another example embodiment includes a tissue extraction device. The tissue extraction device includes a first handle, the first handle including a first guide and terminating in a first tip. The first tip includes denticulations. The tissue extraction device also includes a second handle. The second handle is coupled to the first handle and terminates in a second tip, where the second tip includes denticulations. The second handle includes a second guide configured to mate with the first guide. The tissue extraction device further includes a tissue collection mechanism, where the tissue collection mechanism is configured to collect tissue extracted from a patient by the first tip and the second tip. The tissue extraction device additionally includes an air line connected to the tissue collection mechanism.

These and other objects and features of the present invention will become more fully apparent from the following description and appended claims, or may be learned by the practice of the invention as set forth hereinafter.

### BRIEF DESCRIPTION OF THE DRAWINGS

To further clarify various aspects of some example embodiments of the present invention, a more particular description of the invention will be rendered by reference to specific embodiments thereof which are illustrated in the appended drawings. It is appreciated that these drawings depict only illustrated embodiments of the invention and are therefore not to be considered limiting of its scope. The invention will be described and explained with additional specificity and detail through the use of the accompanying drawings in which:

FIG. 1A illustrates a top perspective view of an example of a tissue extraction device in an open position;

FIG. 1B illustrates a side view of an example of a tissue extraction device in an open position;

FIG. 2A illustrates a close up view of a proximal portion of the tissue extraction device of FIGS. 1A and 1B in an open position;

FIG. 2B illustrates a close up view of a proximal portion of the tissue extraction device of FIGS. 1A and 1B in a closed position;

FIG. 3A illustrates a top perspective view of an example of a first member and a second member coupled at a common pivot point;

FIG. 3B illustrates a side view of an example of a first member and a second member coupled at a common pivot point;

FIG. 4 illustrates an example of an alternative tissue extraction device in a closed position;

FIG. 5A illustrates an example of a tissue extraction device including a ratcheting locking system;

FIG. 5B illustrates an example of a tissue extraction device including a single guide mechanism;

FIG. 5C illustrates an example of a tissue extraction device including a double guide mechanism;

FIG. 6 illustrates an example of an alternative tissue extraction device;

FIG. 7 illustrates an example of distal members and of an alternative tissue extraction device in a closed position;

FIG. 8 illustrates an example of an alternative tissue extraction device having a groove and vacuum line connection;

FIG. 9 illustrates an example of a tissue extraction device with side seals; and

FIG. 10 illustrates an example of graft collection canister coupled to tissue extraction device.

### DETAILED DESCRIPTION OF SOME EXAMPLE EMBODIMENTS

Reference will now be made to the Figures wherein like structures will be provided with like reference designations. It is understood that the Figures are diagrammatic and schematic representations of some embodiments of the invention, and are not limiting of the present invention, nor are they necessarily drawn to scale.

Although the examples and embodiments described herein refer to extraction of tissue samples taken during a follicular dissection, extraction and transplantation procedure or a “strip hair transplantation” such examples are meant for illustrative purposes and are not intended to limit the scope of the disclosure. The disclosed device and method is useful for collection and or extraction of any of a variety of tissue and/or bone specimens collected during any of a variety of surgical procedures. Such tissues may include skin, collagen, muscle tissue, ligament, tendon, vascular, neural and/or bone graft. Additionally, such surgical procedures may include biopsy, cosmetic plastic surgery, reconstructive burn surgery and Anterior Cruciate Ligament (ACL) surgery, for example.

In various embodiments, a tissue extraction device described herein may comprise a variety of materials such as, for example, polymeric materials such as thermoplastics, thermosets, and/or elastomers, and/or metals such as, for instance, stainless steel, titanium and/or aluminum. The material may be transparent, opaque or a combination thereof. The material maybe resistant to wear and/or Food and Drug Administration (FDA) compliant.

A tissue extraction device may be obtained by a variety of processes such as, for instance, machining, stamping, molding and/or vulcanizing processes. Various features of a tissue extraction device may comprise different materials. For example, a hand held end portion may comprise plastic materials such as polycarbonate or isoprene and gripping/clamping end may be made of a different material, such as, for instance, metal ceramic and/or thermoplastic.

In various hair transplant procedures, follicular units may be dissected with punches. After a punch dissects the tissue surrounding a hair follicle, a first end portion of a tissue extraction device may be inserted around a follicular unit. A tissue extraction device may grasp, grip, clamp and/or hold a follicle, pull it out and release it. By using such extractors, such as a tissue extraction device, a single graft may be pulled out and/or multiple grafts may be collected one after the other in the collection area just behind the tip. After the grafts are pulled out, the device may have a vacuum collection system to collect each graft or multiple grafts in a collection chamber or directly connected to a graft organizing station (not shown). The advantages of these designs are simplicity of use, reduction of down time, possibility of collecting multiple grafts and vacuum assisted graft collection.

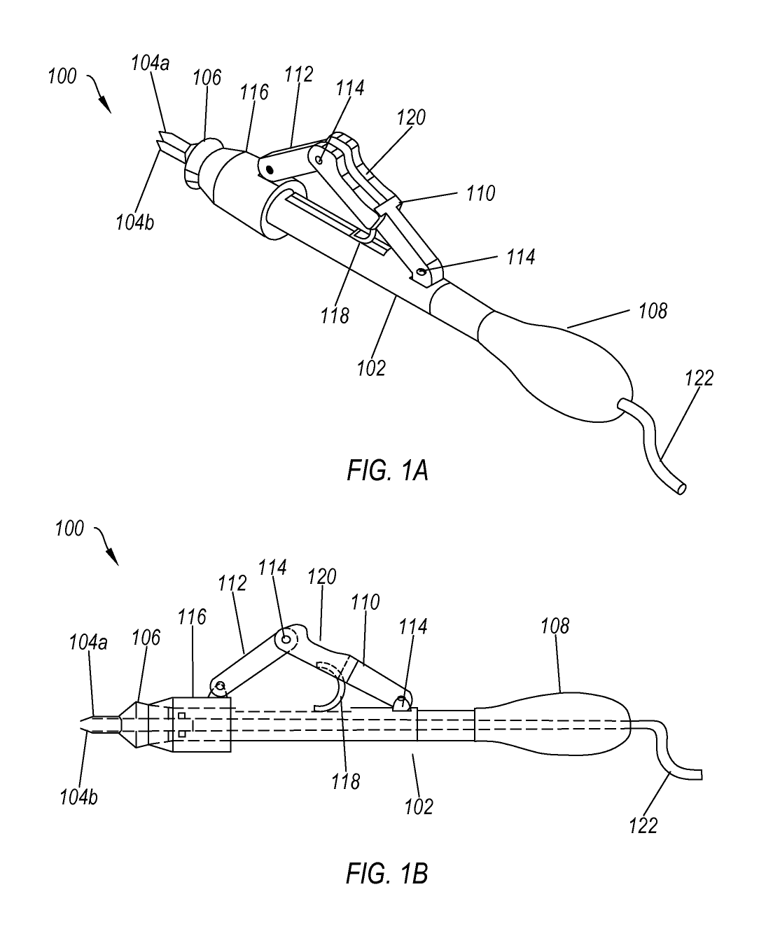

FIGS. 1A and 1B illustrates an example of a tissue extraction device 100 in an open position. FIG. 1A illustrates a top perspective view of an example of a tissue extraction device 100 in an open position; and FIG. 1B illustrates a side view of an example of a tissue extraction device 100 in an open position. Tissue extraction device 100 may be used in a follicular dissection procedure, for instance, for extracting hair follicles.

FIGS. 1A and 1B show that the tissue extraction device 100 can include a main member 102. The main member 102 includes a body which is capable of supporting the other portions of the tissue extraction device 100. In particular, the main member is configured to support tips 104a and 104b, tapered surface 106, handle 108, first linkage 110 and second linkage 112 supported with pins 114, sleeve 116 and spring 118.

FIGS. 1A and 1B also show that the tissue extraction device 100 can include a handle 108, first linkage 110, second linkage 112, and sleeve 116 which may be coupled to each other via pins 114. During use, pins 114 may allow rotational motion relative to connected parts. When first linkage 110 is projected forward by user (not shown) via thumb rest 120, it may compress spring 118 coupled to linkage 110. Spring 118 may be fixedly coupled to the main member and slide against surface of member 110. Sleeve 116 may come into contact with tapered surface 106 on the back of tips 104a and 104b. Tips 104a and 104b close towards each other with a clamping motion. When a user's thumb is removed, sleeve 116 may return as spring 118 returns to non-compressed length. As a result, tips 104a and 104b spread out to the initial position.

Air line 122 may include a vacuum line directly connected to a graft organizing station (not shown) where the collected grafts undergo further processing before they are placed. Additionally or alternatively, air line 122 may be connected to a compressed air line. A switch like a solenoid valve switch operated by hand or foot (not shown) may be used to activate either the compressed air or vacuum. The air line 122 may be used to collect the grafts in to a canister (see FIG. 10) while the compressed air may be used to remove the grafts from the collection area.

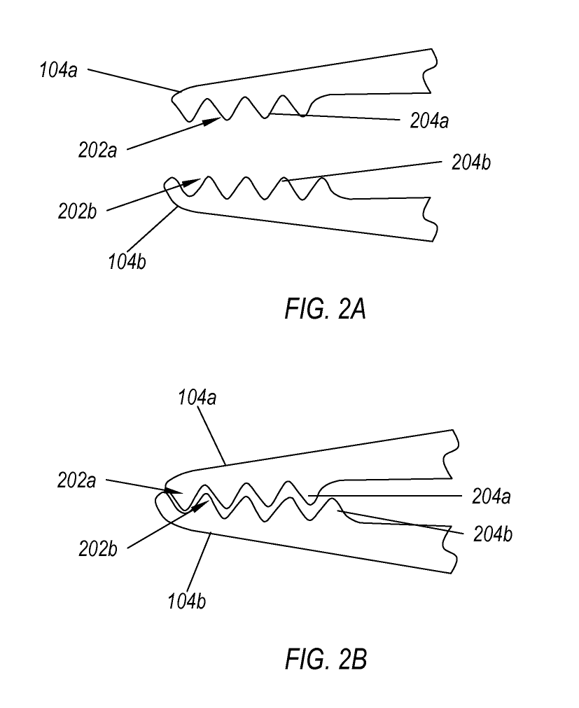

FIGS. 2A and 2B illustrate a close-up view of a proximal portion of the tissue extraction device 100 of FIGS. 1A and 1B. FIG. 2A illustrates a close up view of a proximal portion of the tissue extraction device 100 of FIGS. 1A and 1B in an open position; and FIG. 2B illustrates a close up view of a proximal portion of the tissue extraction device 100 of FIGS. 1A and 1B in a closed position. The first tip 104a may comprise a first gripping inner surface 202a comprising first surface structure 204a capable of gripping tissues and second tip 104b may comprise a second gripping inner surface 202b comprising second surface structure 204b capable of gripping tissues. Tips 104a and 104b may move close to or apart from each other, when user imparts a closing or an opening force. The surface structures 204a and 204b shown may be denticulation profiles and may include arcs, lines and or other geometric shapes. The first surface structure 204a on first gripping surface 202a may be capable of mating or interlocking with the second surface structure 204b on second gripping surface 202b. In a closed position shown in FIG. 2B, the separation between the first tip 104a and second tip 104b may be between 0.1 mm and 5 mm.

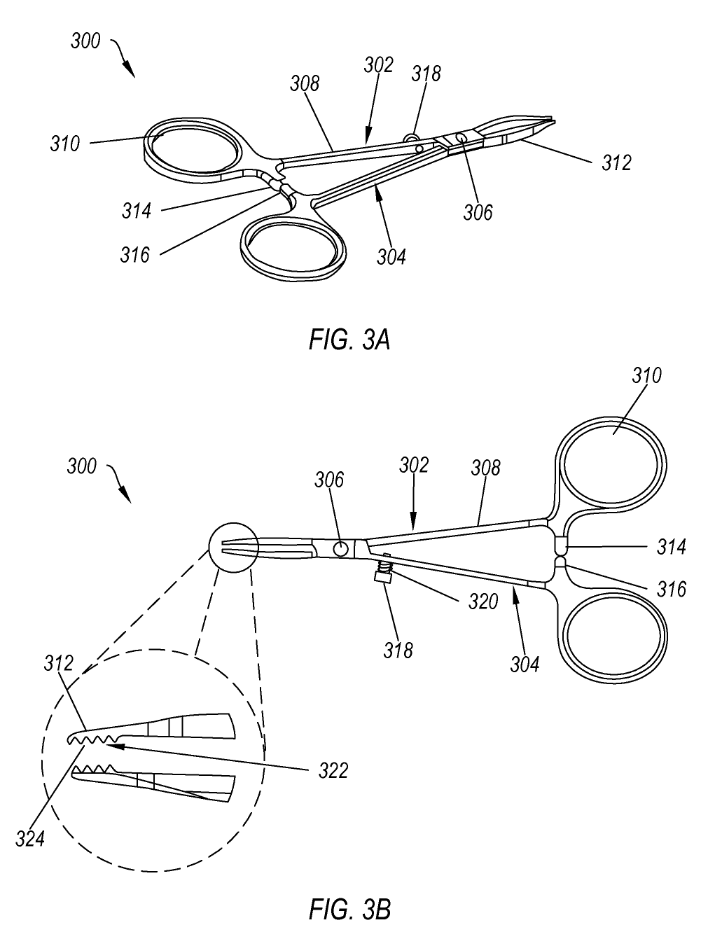

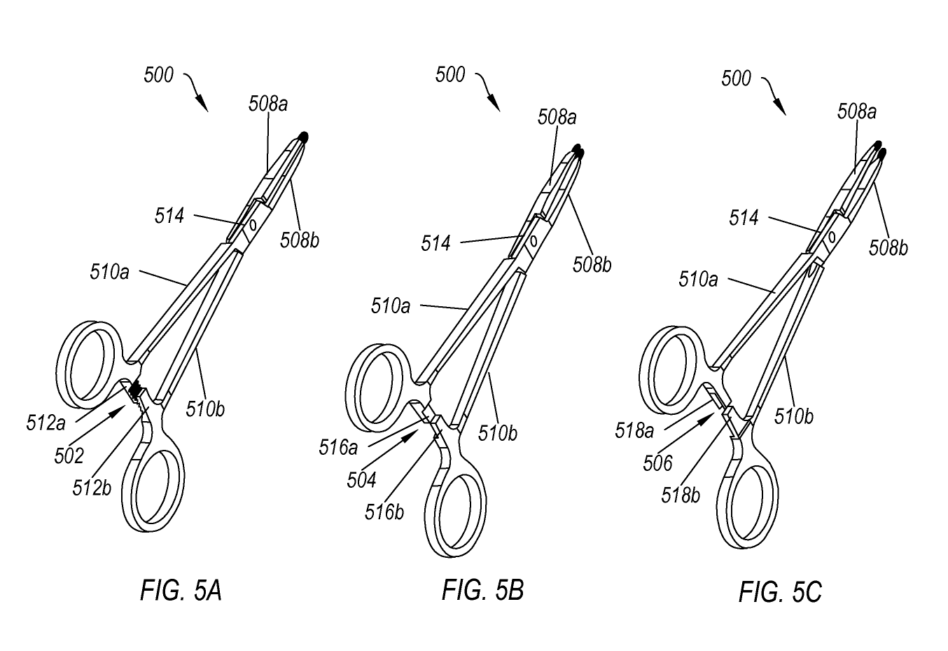

FIGS. 3A and 3B illustrates an example of a first member 302 and a second member 304 coupled at a common pivot point 306. FIG. 3A illustrates a top perspective view of an example of a first member 302 and a second member 304 coupled at a common pivot point 306; and FIG. 3B illustrates a side view of an example of a first member 302 and a second member 304 coupled at a common pivot point 306. Pivot point 306 may comprise any of a variety of coupling devices such as a pin, screw, hinge, spring and/or clip. First member 302 may comprise a first lever portion 308, a first finger hole 310, first distal jaw portion 312 and a first guide 314. The first lever portion 308 may have a variety of lengths and shapes. For example, the first lever portion 308 may be between 20 mm and 75 mm in length and may comprise a substantially straight shape. The first finger hole 310 may be positioned at a proximal portion of first lever portion 308 such that it may be capable of receiving a user's fingers for manual operation of tissue extraction device 300. Also, first finger hole 310 may include a variety of materials, shapes and diameters. For example, first finger hole 310 may be substantially round and may have a diameter of between 15 mm and 35 mm. Additionally, first guide 314 may comprise a variety of mating structures and may be capable of mating to second guide 316. Such mating structures are discussed below with regard to FIGS. 5A, 5B and 5C.

FIGS. 3A and 3B show that a screw 318 and a spring 320 can be positioned on second member 302 of the tissue extraction device 300 and may be used as a stopper to limit the downward movement of member 304 of the tissue extraction device 300 to limit the force applied on the tissue at the first distal jaw portion 312 when applied by the user.

FIGS. 3A and 3B also show that first distal jaw portion 312 may comprise a first gripping inner surface 322 comprising surface structures 324 capable of gripping tissues. First distal jaw portion 312 may be pushed or pulled by the first lever portion 308 at proximal ends of the first member 302 in order to move first distal jaw portion 312 from an open position to a closed position. First distal jaw portion 312 may move close to or apart from opposing jaw portion, when user imparts a closing or an opening force at the proximal end of the first member 302. First distal jaw portion 312 may be shaped in such a way that in the open mode, it can be adjusted and inserted into a hole around a follicle without affecting the cut follicle. First distal jaw portion 312 may have inner serrated face with profiles disposed towards the tip of First distal jaw portion 312 for gripping the graft in between the jaws (not shown).

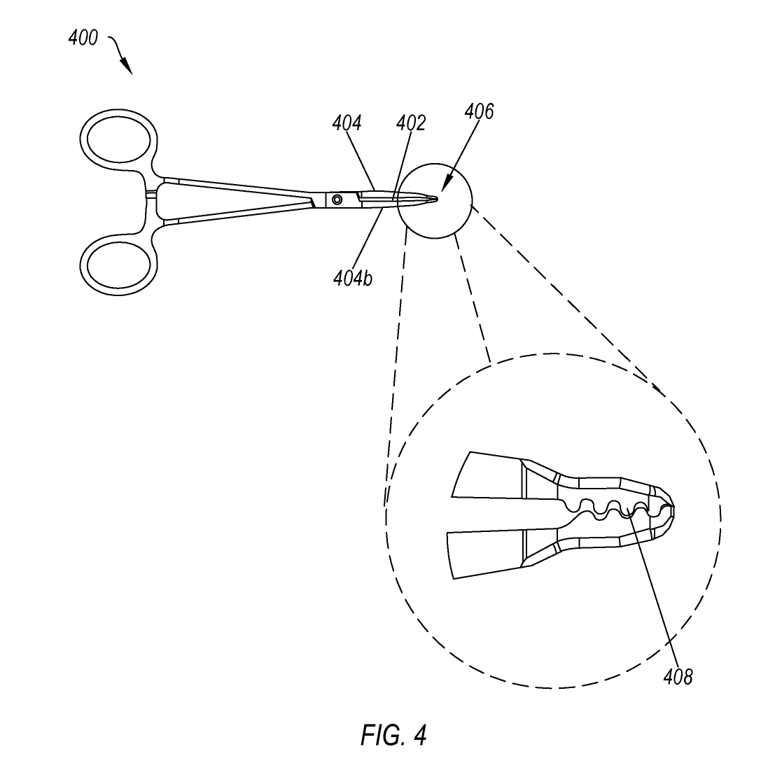

FIG. 4 illustrates an example of an alternative tissue extraction device 400 in a closed position. Clearance 402 between jaws 404a and 404b is shaped in such a way as to provide room or space behind the denticulations to collect grafts temporarily. I.e., the geometry of tip 406 and clearance 402 are designed to collect multiple grafts (not shown). The two jaws 404a and 404b of the tissue extraction device 400 in a partially open position are inserted into a dissected hole around a graft. The jaws 404a and 404b may be closed to grip the graft then pulled out of the hole. To extract another graft, the tissue extraction device 400 is partially opened again and pushed into a new hole with a graft where the already collected graft between the teeth may be displaced backwards into space 402 by the new graft. Thus, newly collected grafts may push the preceding grafts towards the back into the clearance area 402. All preceding grafts may be stored in clearance area 402 of tissue extraction device 400. Additionally or alternatively, the collected grafts may be removed and stored in a solution to clear space for further extraction.

FIG. 4 shows that, during a follicular extraction procedure, as grafts are being extracted, a newly extracted graft (not shown) may push a preceding graft (not shown) back into clearance 402. As newly removed grafts push one another backwards and are collected in clearance 402. A large number of grafts, up to about 50, may be collected one after the other without any damage and without having to move from the harvest site to another graft collection area. Teeth 412 may be positioned such that they are mated in order to enable gripping of follicular graft without damaging the graft.

FIGS. 5A, 5B and 5C illustrate examples of an alternative tissue extraction device 500 including various guide systems. FIG. 5A illustrates an example of a tissue extraction device 500 including a ratcheting locking system 502; FIG. 5B illustrates an example of a tissue extraction device 500 including a single guide mechanism 504; and FIG. 5C illustrates an example of a tissue extraction device 500 including a double guide mechanism 506. The guide systems can ensure that the closing of the handles of the tissue extraction device 500 move in a known way relative to one another. Additionally or alternatively, the guide systems can ensure that the handles of the tissue extraction device 500 remain in a known position relative to one another.

FIG. 5A shows that the tissue extraction device 500 can include a ratchet locking mechanism 502 to enable gripping, locking and/or alignment of handles 508a and 508b. Leverage bars 510a and 510b are disposed with locking features 512a and 512b. In addition, mating surfaces 514 may keep handles 508a and 508b aligned and/or locked together.

FIG. 5B shows that the tissue extraction device 500 can include a single guide mechanism 504. The tissue extraction device 500 may have a single guide system 504 to enable gripping, locking and/or alignment of handles 508a and 508b. Leverage bars 510a and 510b are disposed with guide features 516a and 516b to keep handles 508a and 508b aligned and/or locked together. In addition, mating surfaces 514 of handles 508a and 508b may help to align and guide handles 508a and 508b.

FIG. 5C shows that the tissue extraction device 500 can include a double guide mechanism 506. The tissue extraction device 500 may have a double guide mechanism 506 to enable gripping, locking and/or alignment of handles 508a and 508b. Leverage bars 510a and 510b are disposed with guide features 518a and 518b to keep handles 508a and 508b aligned and/or locked together. In addition, mating surfaces 514 of handles 508a and 508b may help to align and guide handles 508a and 508b.

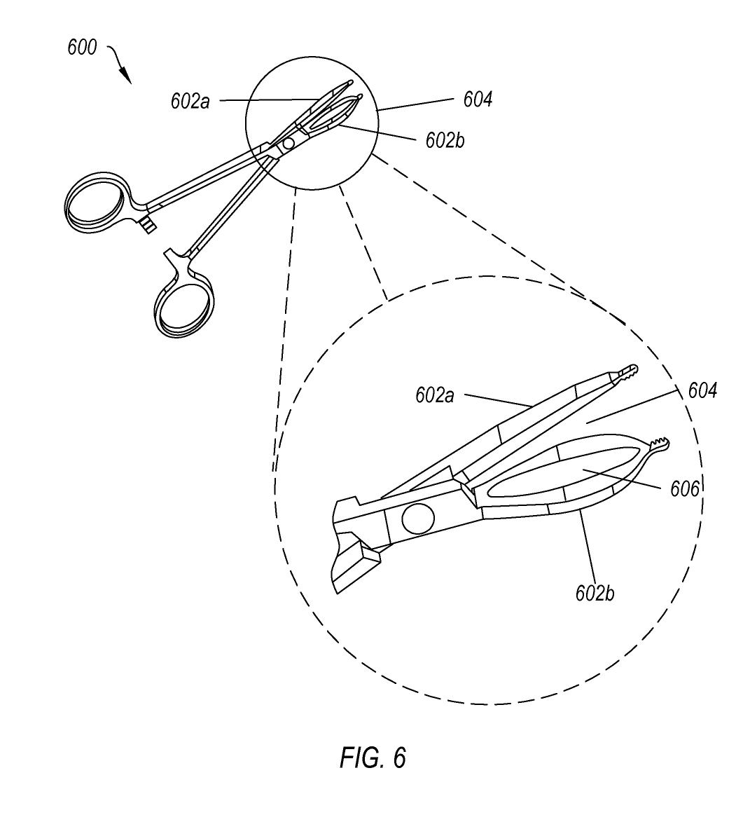

FIG. 6 illustrates an example of an alternative tissue extraction device 600. The tissue extraction device 600 may be capable of collecting multiple grafts. The tissue extraction device 600 may have a first jaw portion 602a and a second jaw portion 602b. The second jaw portion 602b may be made wider that first jaw portion 602a in order to collect a large number of grafts (e.g., up to about 50). A graft may be collected at a site and may be stored in tissue extraction device 600 during extraction of other grafts. As a follicular dissection procedure progresses extracted grafts (not shown) push the preceding graft back into clearance 604.

As an example, grafts are removed and abut one another. Grafts may then be collected on the wider surface area of the jaws. Collection of grafts may be accomplished without any damage and without having to move from the harvest site to another graft collection area at each dissection cycle.

FIG. 6 also shows that groove 606 is also disposed at center of inner side 608 of second jaw portion 602b. Groove 606 may guide grafts (not shown) towards center to prevent grafts from falling out at the edges. Grafts that were extracted from the donor site and temporarily collected in the space between the jaws behind the denticulations may be removed from tissue extraction device 600 and preserved until the end of a follicular dissection procedure. The grafts may be removed by hand or blown with a compressed air into a petri dish containing a storage solution like BSS or hypothermosol.

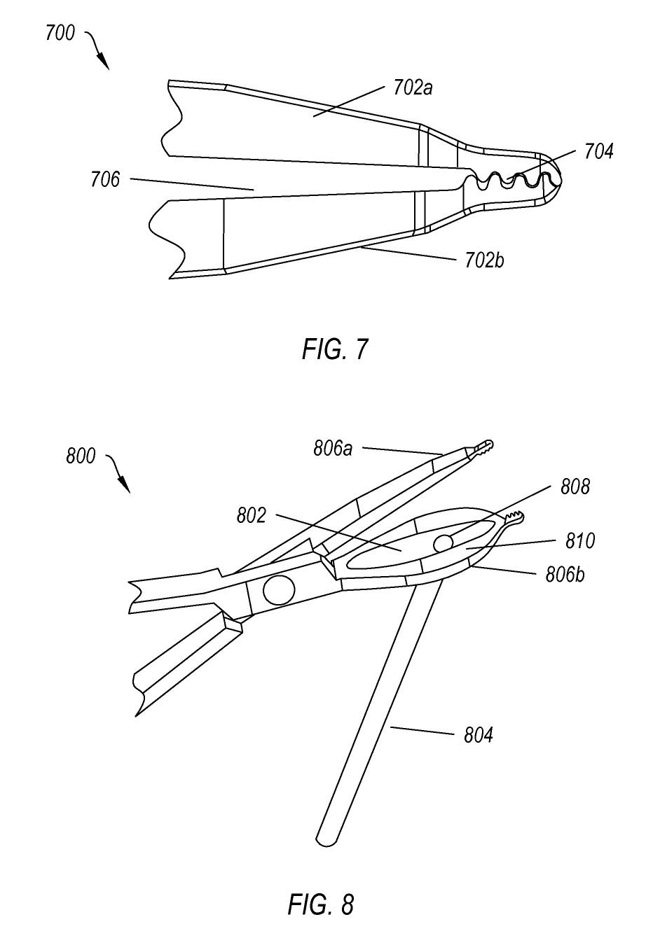

FIG. 7 illustrates an example of distal members 702a and 702b of an alternative tissue extraction device 700 in a closed position. Teeth 704 may be positioned such that they are mated in order to enable gripping of follicular graft. Teeth 704 may be any of a variety of mating surfaces such as, for instance, grooves, serrations or denticulations. Clearance 706 may allow multiple grafts to be extracted without having to stop to remove grafts from tissue extraction device 700.

FIG. 8 illustrates an example of an alternative tissue extraction device 800 having a groove 802 and vacuum line connection 804. Groove 802 is disposed at center of second jaw portion 806b. Groove 802 may guide grafts (not shown) towards the center. Additionally or alternatively, a void 808 may be disposed inside groove 802 on inner surface 810 of second jaw portion 806b. Void 808 may connect a cavity (not shown) formed by the inner surfaces of first jaw portion 806a and second jaw portion 806b to vacuum line 804. A hose line from a vacuum system (not shown) may be connected to line 804.

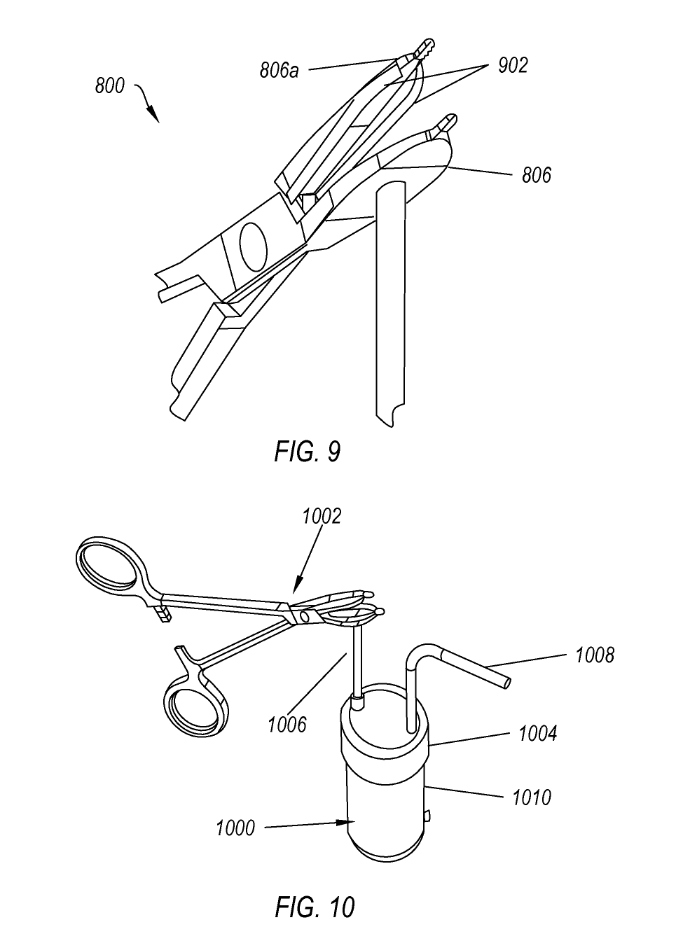

FIG. 9 illustrates an example of a tissue extraction device 800 with side seals 902. Side seals 902 may be applied to first jaw portion 806a and/or second jaw portion 806b to increase the effectiveness of the vacuum system. Side seals 902 may be made of a variety of substances such as rubber and/or silicon. When the jaws are closed, the upper and lower jaws and the side seals 902 create an enclosure or cavity. This enclosure may assist the vacuum graft removal process by creating a tight seal between first jaw portion 806a and second jaw portion 806b.

FIG. 10 illustrates an example of graft collection canister 1000 coupled to tissue extraction device 1002 and vacuum pump (not shown). Graft collection canister 1000 may comprise cap 1004 connected to incoming line 1006 and outgoing line 1008. Cap 1004 may fit snugly over a bottom portion 1010 of graft collection canister 1000. Bottom portion 1010 of graft collection container 1000 may be partially filled with an appropriate solution (such as Saline solution, BSS solution Hypothermosol) to hydrate and/or preserve the grafts. During a follicular isolation procedure, bottom portion 1010 of graft collection canister 1000 containing the grafts may be removed and replaced with an empty one without disconnecting the incoming line 1006 or outgoing line 1008.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A tissue extraction device, the tissue extraction device comprising:
a first handle, the first handle terminating in a first tip;
a second handle, the second handle:
coupled to the first handle; and
terminating in a second tip; and
a tissue collection mechanism, wherein the tissue collection mechanism is configured to collect tissue extracted from a patient by the first tip and the second tip.

2. The tissue extraction device of claim 1, wherein the tissue collection mechanism includes a clearance configured to store the extracted tissue.

3. The tissue extraction device of claim 1, wherein the clearance between the first tip and the second tip in a closed position is between 0.1 mm and 5 mm.

4. The tissue extraction device of claim 1, wherein the first tip includes denticulations.

5. The tissue extraction device of claim 4, wherein the second tip includes denticulations.

6. The tissue extraction device of claim 4, wherein the number of denticulations is between 2 denticulations and 10 denticulations.

7. The tissue extraction device of claim 5, wherein the first tip is configured to mate with the second tip.

8. The tissue extraction device of claim 1, wherein the first handle includes a guide.

9. The tissue extraction device of claim 8, wherein the second handle includes a second guide configured to mate with the guide.

10. A tissue extraction device, the tissue extraction device comprising:
a first handle, the first handle:
terminating in a first tip, wherein the first tip includes denticulations; and
including a first guide;
a second handle, the second handle:
coupled to the first handle;
terminating in a second tip, wherein the second tip includes denticulations; and
includes a second guide configured to mate with the first guide; and
a tissue collection mechanism, wherein the tissue collection mechanism is configured to collect tissue extracted from a patient by the first tip and the second tip.

11. The tissue extraction device of claim 10, wherein the first handle is coupled to the second handle by a hinge.

12. The tissue extraction device of claim 10, wherein the first handle is coupled to the second handle by a pin.

13. The tissue extraction device of claim 10, wherein the first and second guide includes ratchet members.

14. The tissue extraction device of claim 10, wherein the first tip has a larger profile than the second tip.

15. A tissue extraction device, the tissue extraction device comprising:
a first handle, the first handle:
terminating in a first tip, wherein the first tip includes denticulations; and
including a first guide;
a second handle, the second handle:
coupled to the first handle;
terminating in a second tip, wherein the second tip includes denticulations; and
includes a second guide configured to mate with the first guide;
a tissue collection mechanism, wherein the tissue collection mechanism is configured to collect tissue extracted from a patient by the first tip and the second tip; and
an air line connected to the tissue collection mechanism.

16. The tissue extraction device of claim 15, wherein the tissue collection mechanism includes a void.

17. The tissue extraction device of claim 15, wherein the air line includes a vacuum line.

18. The tissue extraction device of claim 17 further comprising a graft collection canister coupled to the vacuum line.

19. The tissue extraction device of claim 18 further comprising a solution within the collection canister.

20. The tissue extraction device of claim 15, wherein the air line includes a compressed air line.
